(19) United States
(12) Patent Application Publication
(10) Pub. No.: US 2026/0256496 A1
(43) Pub. Date: Sep. 3, 2026

(54) CERCLAGE DEVICE AND METHOD FOR PREVENTING PREMATURE BIRTH

(71) Applicant: MOR RESEARCH APPLICATIONS LTD., Ramat Gan (IL)

(72) Inventors: Polina SCHVARZMAN, Ramat Gan (IL); Idan TRAISTMAN, Ramat Gan (IL)

(21) Appl. No.: 18/994,631

(22) PCT Filed: Aug. 9, 2023
(86) PCT No.: PCT/IB2023/058056

§ 371 Date: Jan. 15, 2025

Related U.S. Application Data
(60) Provisional application No. 63/397,122, filed on Aug. 11, 2022.

(51) Int. Cl. A61B 17/42; A61B 17/12

(52) U.S. Cl. CPC A61B 17/42; A61B 17/12009; A61B 2017/4225

(57) ABSTRACT

Aspects of embodiments provides devices and methods for cervical locking during cerclage to prevent miscarriage or premature delivery, the device comprising a passage to guide a suture therethrough and enable closure thereof around a distal end of a cervix in a female subject, the device adapted to remain engaged around the cervix over a period of time until the suture is cut, thereby preventing miscarriage or premature delivery.

### CROSS-REFERENCE TO RELATED APPLICATIONS

This patent application claims priority and benefit from U.S. provisional application 63/397,122, filed Aug. 11, 2022, titled “CERCLAGE DEVICE AND METHOD FOR PREVENTING PREMATURE BIRTH”, and which is incorporated herein by reference in its entirety.

### BACKGROUND

Cervical insufficiency (cervical incompetence) is reported as 0.5% in the general obstetric population and 8% in women with a history of previous mid-trimester miscarriages. This condition is defined as a painless opening and dilatation of the uterine cervix, leading to late miscarriage or preterm delivery.

In order to prevent these type of miscarriage or preterm delivery, a gynecological procedure, known as cervical cerclage, was developed in the 1950s-1960s.

In cervical cerclage, a stitch, such as a purse-string stitch is performed by a surgeon to force shut the cervix. This stitching is usually performed in 4 points throw the cervix and includes a suture band knot to an upper region of the cervix. Typically, cerclage is performed around 13-14 week of gestation under anesthetic and is removed after 37 week, or earlier if required. Usually, removal is office procedure without anesthesia.

There are three types of cerclage:
1. McDonald cerclage, described in 1957, is the most common, placed to the cervix via vaginal approach, with knot leaved protruding to the vagina.
2. A Shirodkar cerclage is very similar, but the sutures pass through the walls of the cervix so they're not exposed, the knot may be closed by vaginal tissue or leaved inside the vagina in Modified Shirodkar cerclage
3. An abdominal cerclage, the least common type, is permanent and involves placing a band at the very top and outside of the cervix, inside the abdomen. This is usually only done if the cervix is too short to attempt a standard cerclage, or if a vaginal cerclage has failed or is not possible. This type of cerclage must be removed in some cases, for instance, when pregnancy is pathologic, and curettage is needed

One problem associated with the cerclage procedure is that the stitch may be ingrained into the tissue, as a consequence, may be difficult to find the region for cutting in order to remove it. When the knot is embedded to vaginal tissue the removal may cause a significant discomfort for the patient and moreover, it may lead to cervical damage.

There therefore remains a need to provide improved cervical cerclage devices and methods.

### BRIEF DESCRIPTION OF THE DRAWINGS

With specific reference now to the figures in detail, it is stressed that the particulars shown are by way of example and for purposes of illustrative discussion of the embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1D are simplified perspective pictorial illustrations showing a cerclage device for preventing a miscarriage, in accordance with embodiments;

FIGS. 2A-2D are further simplified perspective pictorial illustrations showing a cerclage device for preventing a miscarriage, in accordance with embodiments;

FIG. 3 is a simplified flow chart of a method for performing cervical cerclage, in accordance with an embodiment of the present invention;

FIG. 4A is a simplified transverse illustration of an open device inserted to engage a distal end of a cervix, in accordance with an embodiment.

FIG. 4B is a simplified transverse illustration of the closed (knotted) device inserted to engage a distal end of a cervix, in accordance with an embodiment.

FIG. 4C is a simplified cross-sectional illustration of an open device, ready for use, in accordance with an embodiment.

FIG. 4D is simplified schematic illustrations showing a lower view, looking upwards of closed cerclage device inserted to engage a distal end of a cervix, for preventing a miscarriage, in accordance with embodiments.

FIG. 5A is a simplified transverse illustration of an open device inserted to engage a distal end of a cervix, in accordance with an embodiment.

FIG. 5B is a simplified transverse illustration of the closed (knotted) device inserted to engage a distal end of a cervix, in accordance with an embodiment.

FIG. 5C is a simplified cross-sectional illustration of an open device, ready for use, in accordance with an embodiment.

FIG. 5D is another simplified cross-sectional illustration of an open device, ready for use, in accordance with an embodiment.

### DETAILED DESCRIPTION

Aspects of embodiments relates generally to methods and devices for preventing premature births, including cervix cerclage methods and devices.

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

It is an object of some embodiments of the present invention to provide improved cervical cerclage methods and devices.

In some embodiments of the present invention, improved methods and devices are provided for cervical cerclage.

In some further embodiments of the present invention, improved methods and devices are provided for preventing miscarriage.

In some further embodiments of the present invention, improved methods and devices are provided for preserving pregnancies.

In some embodiments of the present invention, improved methods and devices are provided for locking during cerclage (e.g., cervical cerclage).

According to some embodiments, the device is constructed and configured to:
a) be introduced to a vagina and of a female before or during a pregnancy;
b) provide a suture or suture band that encompasses the cervix and keeps it substantially shut to prevent miscarriage of at least one fetus;
c) enable easy cutting of the suture after the period of time; and
d) removal of the device before birth of the at least one fetus.

According to some embodiments of the present invention, the device is constructed and configured to
a) be introduced to a vagina and of a human female at a first point in time before or during a pregnancy;
b) provide a suture or suture band that encompasses the cervix and keeps it substantially locked shut to prevent miscarriage of at least one fetus;
c) remain in place until the at least one fetus is generally viable;
d) enable easy cutting of the suture at a second point in time in the pregnancy; and
e) removal of the device and substantially all suture material before birth of the at least one fetus.

According to some embodiments, the device is constructed and configured to:
a) be introduced to a vagina of a pregnant female around an end of a first trimester of the pregnancy;
b) provide a suture or suture band that encompasses the cervix in tight fit;
c) remain in place until the fetus is generally viable;
d) enable easy cutting of the suture;
e) removal of the device and the suture before birth of the fetus.

According to some embodiments, the device is constructed and configured to:
a) be introduced into a vagina of a pregnant female around an end of a first trimester of the pregnancy;
b) provide a suture or suture band that encompasses the cervix in tight fit;
c) remain in place for a period of time;
d) enable easy cutting of the suture after the period of time;
e) removal of the device and the suture material.

According to some embodiments, the device is constructed and configured to:
a) be introduced into a vagina of a pregnant female around an end of a first trimester of the pregnancy;
b) provide a suture band that encompasses the cervix;
c) adjust tightening element to tighten the suture band around the cervix;
d) remain in place for a period of time;
e) enable easy cutting of the suture band after the period of time;
f) removal of the device and the suture material.

According to some embodiments, the device has passage to guide a suture and the device remains engaged until suture is cut.

Moreover, the device has a recess due to the which, the suture can be found and cut very easily. In some examples, the devices ensures that the thread is not entirely or almost entirely buried or ingrained in the cervical tissue over time. Due to the device's configuration, a portion of the thread remains non-buried or exposed and, thus, easily accessible by a medical professional (e.g., physician, nurse) for easy access of the thread portion and cutting, at the exposed portion, the thread in half.

Embodiments of the present invention will be more fully understood from the following detailed description of the embodiments thereof, taken together with the drawings.

Cervical cerclage is suggested for singleton pregnancies in women:
With a history of three or more previous mid-trimester losses or premature (<28 week) births
Who have had cervical sutures in previous pregnancies
With history of mid-trimester loss or premature birth with a shortened cervix (<25 mm)
Who are deemed at risk of preterm birth at the clinician's discretion

Multifilament/braided sutures such as Mersilene tape have been traditionally used for cervical cerclage. Braided sutures, particularly mesh-like non-absorbable sutures, have been associated with an increased risk of infection and, hence, some obstetricians may prefer to use monofilament/non-braided sutures.

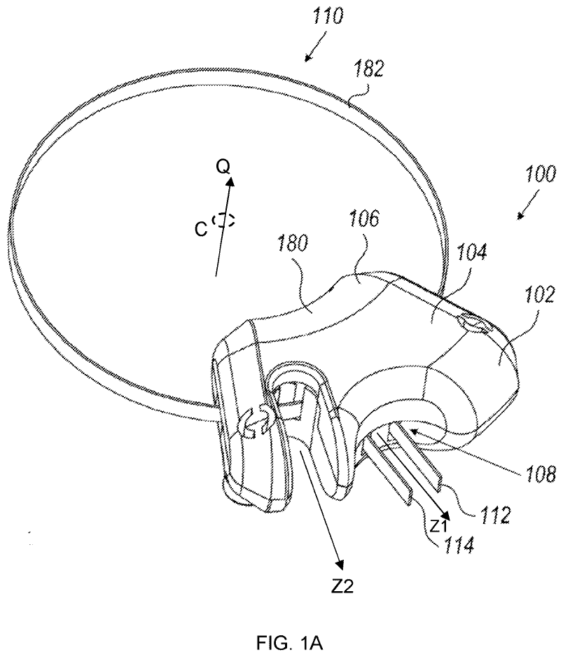

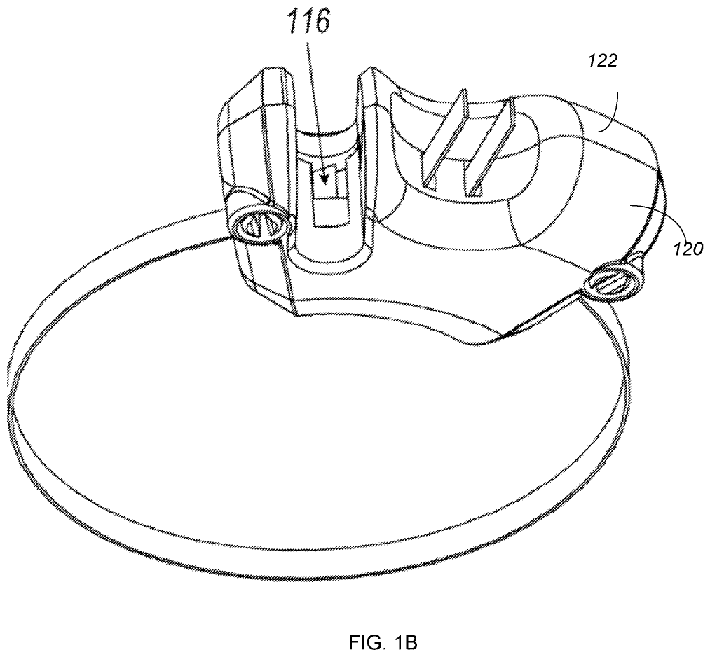

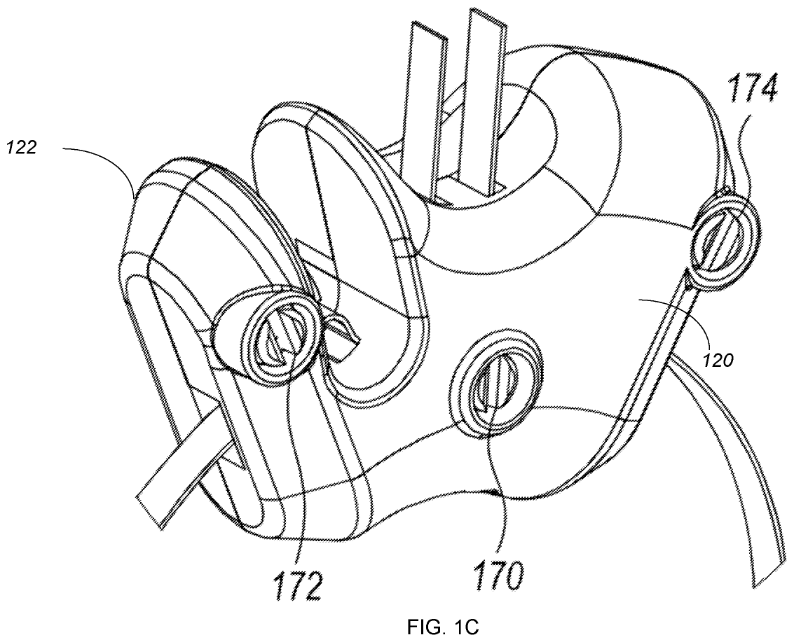

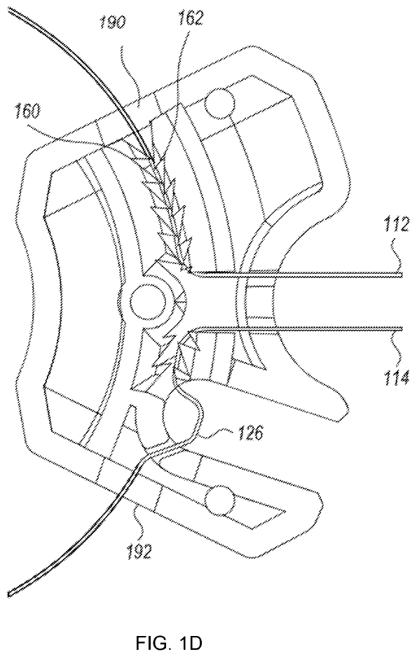

FIGS. 1A-1D are simplified perspective pictorial illustrations showing a cerclage device for preventing a miscarriage, in accordance with embodiments. Turning now to FIG. 1A, there is seen a cerclage suture device 100 having a first recess 108. Suture device 100 is adapted to receive a suture band or thread 110 and to hold to or receive ends thereof 112, 114 within the first recess 108. The device comprises an exterior element 102 (facing away from the cervix), adapted to hold the ends 112, 114 of the thread/suture, a middle element 104 and an interior element 106. By “interior” is meant facing inward towards the cervix. The device 100 may further be configured to have a second recess (also: band cutting recess) 116 for facilitating accessing a portion of the thread 110. In some examples, a device may only have a single recess. In some examples, either the first and/or the second recess may be configured to ensure that a portion of the thread remains exposed (e.g., non-buried in human tissue over time) to facilitate access and cutting of the thread after a prolonged period of time (e.g., after several weeks). Both the first recess 108 and the second recess 116 may be open in an identical or about identical direction Z1 and Z2, pointing away (also: pointing radially in an distal direction of a virtual center C (further away in direction Rd from a longitudinal axis Q of the cervix or vagina) of operably engaged thread 110.

In some embodiments, the three elements 102, 104 and 106 may be coupled with each other (e.g., glued together, screwed together and/or and clasped together with a snap fit and/or other coupling), e.g., so that they cannot be detached from one another without compromising the structural integration of the device. In some other examples, at least two of the external element 102, the middle element 104 and the interior element 106 may be integrally formed with each other.

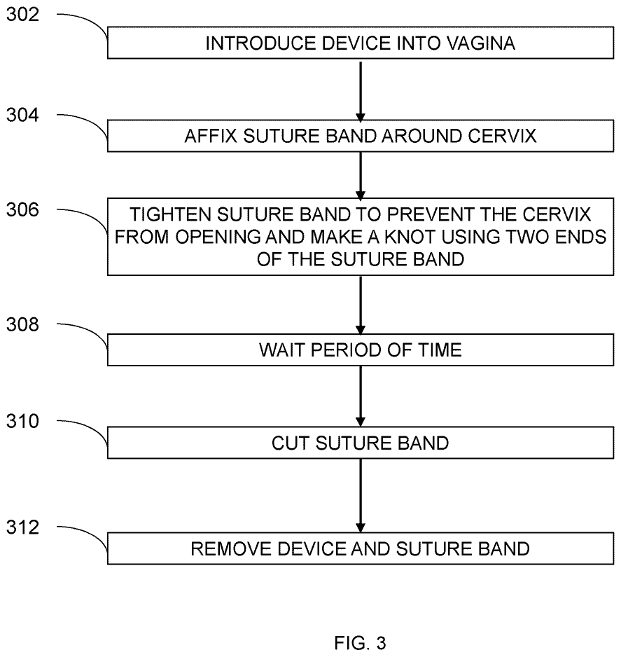

Many types of suture bands and thread can be used with embodiments of the device, as exemplified above. These should not be deemed limiting. One non-limiting example of how devices according to some embodiments can be used is exemplified in FIG. 3, and will be discussed in more detail hereinbelow.

Device 100 includes holding structures for holding the suture band in place, such as a set of lower teeth 160 and a set of upper teeth 162, through which the suture band is threadable. In some examples, the holding structures may be part of the middle element 104.

In some embodiments, the holding structure might include male and female interconnecting or interlocking parts configured to limit the movement/degrees of freedom of the suture band and hold it firmly in place to limit any movement of direction of the longitudinal axis of the suture band and/or transversal to the suture's longitudinal axis. The holding structure may be configured to provide frictional and/or form-limiting coupling or limitation of movement of the thread 110.

In some embodiments, the device may include one or more orifices 170, 172, 174, (FIG. 1C) which are configured to enable operably engaging a tool with the device at various engagement points and/or angles. For example, the surgeon may use a screwdriver for guiding into and/or fastening suture band 110 within the device 100. In some examples, there may be various tightening elements for tightening the grip of the holding structure. The device is constructed and configured to hold a suture band 110 in a good fit, snugly around a cervix.

The device may also comprise one or more orifices 190, 192, through which the suture band is threaded. In some examples, the entire device may be 1-5 cm in length, and 1-3 cm in width and 1-2 cm in depth. The device may be constructed out of any suitable material (e.g., polymeric material, metals, etc.), suitable for inserting into and use within a human body.

In some examples, an inner surface 180 of the inner part is may be arched or concave, serving as a completing a small part of an entire generally circular inner face 182 of the suture band, constructed and configured to be held in place.

In some examples, device 100 may be constructed to have a front cover 120 and a back cover 122, which may be removably coupled with each other allowing exposure of the device's holding structures without compromising the device's structural integrity. Front cover 120 may be opposite back cover 122. Once the holding structures are exposed, the medical professional may operably couple band 110 with the holding structures to arrive at a configuration as for example shown in FIG. 1D. The front and back covers 120 and 122 may be reassembled to obtain the configuration shown for example in FIG. 1C. Hence, instead of threading band 110 through the orifices 190 and 192 for operably coupling band 110 with device 100, band 110 may in some example implementations be transversely pushed into the holding structures. As shown, the configuration of the device facilitates accessing a portion 126 of the thread 110 located in the second recess 116.

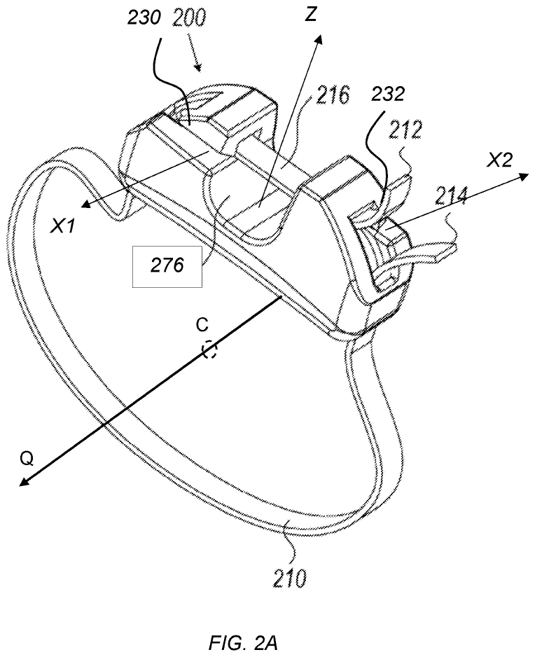

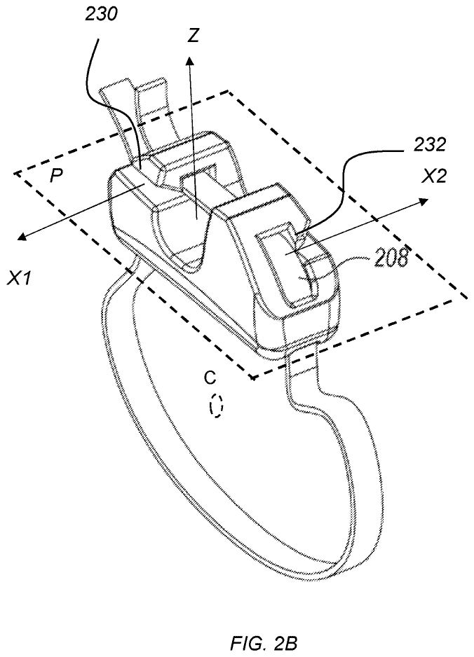

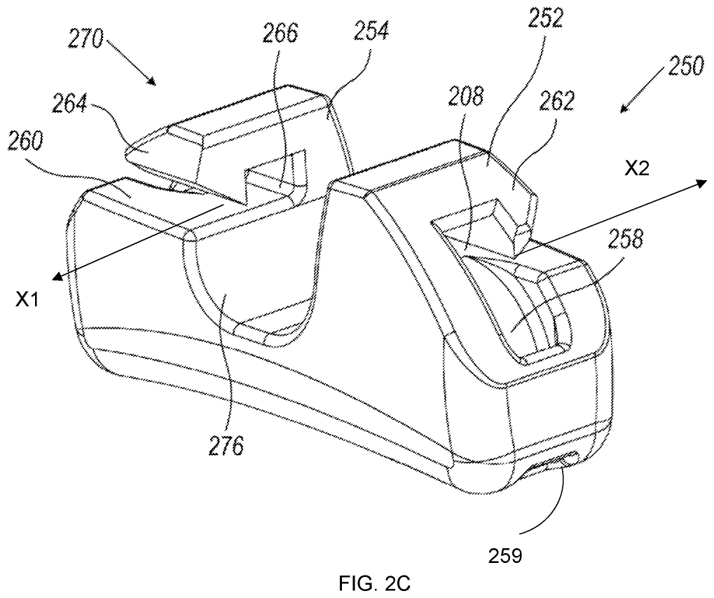

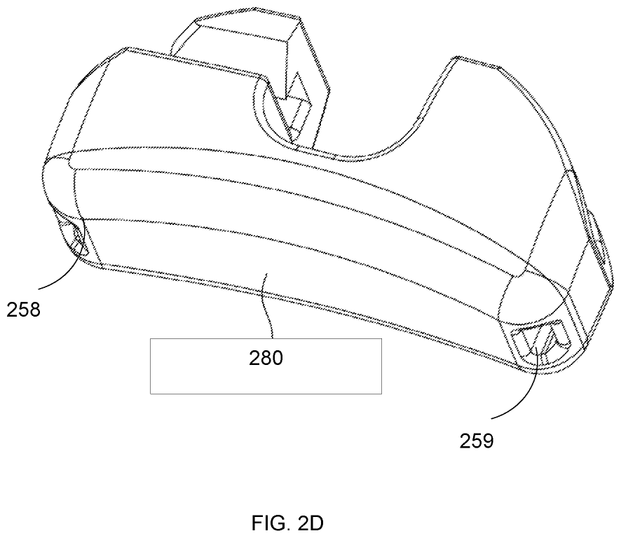

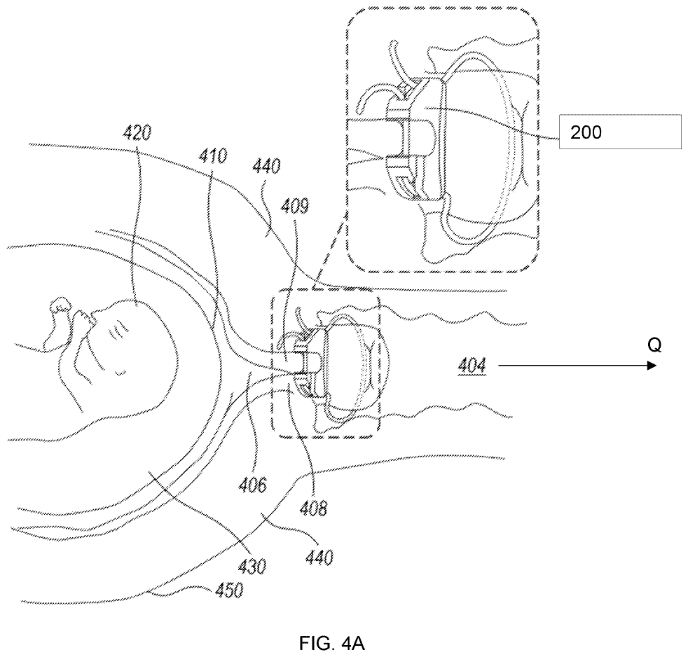

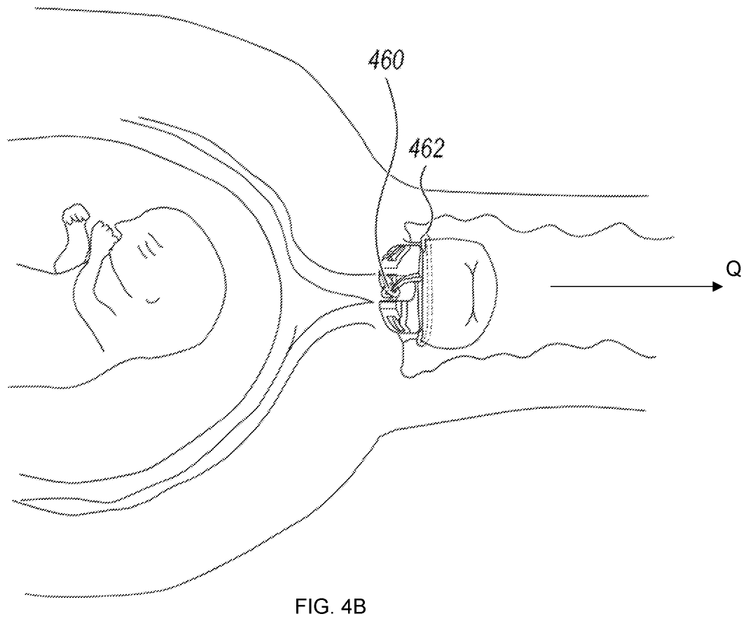

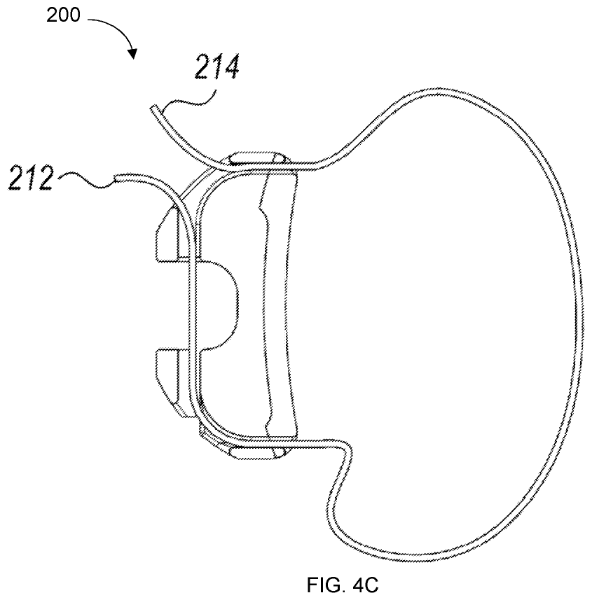

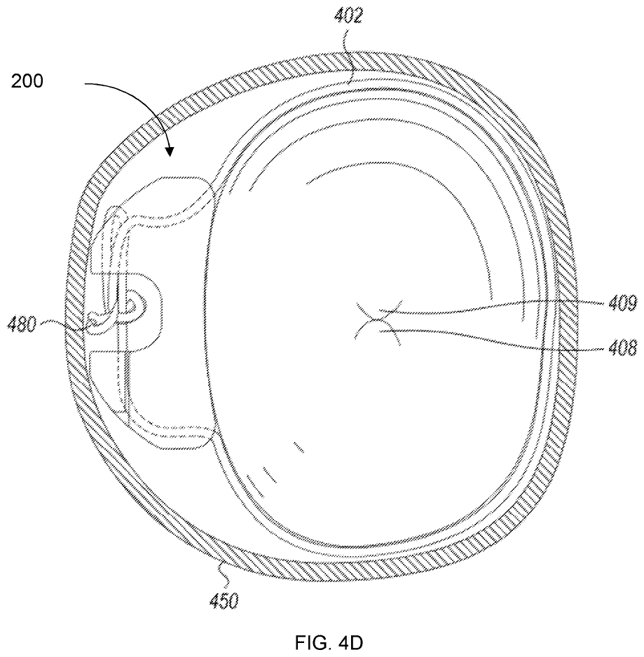

FIGS. 2A-2D are further simplified perspective pictorial illustrations showing a cerclage device 200 for preventing a miscarriage, in accordance with embodiments Device 200 is constructed and configured to hold a suture band 210 in a good fit, snugly around a cervix (see FIGS. 4A-4D). Device 200 may comprise a band cutting recess 276 which facilitates access to a portion of a band 110 used for cervical cerclage. Band cutting recess 276 may be open in a first direction Z, pointing away from a center of the band and lying on a virtual surface that may be delineated by the band when operably engaged with the cervix, optionally analogous to what is described with respect to FIG. 1A.

Band cutting recess 276 recess may be larger than band guiding recesses, and they may be used for cutting an exposed portion 216 of the suture band in order to release the device from the cervix and enable removal thereof from the subject via C-section or from a vagina, as is necessary.

Band cutting recess 276 may be disposed between a first band guiding recess 208 and a second band guiding recess 226. First band guiding recess 208 and second band guiding recess 266 may be open in two main directions X1 and X2 which are opposite to each other and both of which are orthogonal to first direction Z. In some examples, device 200 may be configured such that first band guiding recess 208 and second band guiding recess 226 may aligned with each other, e.g., to lie within a same virtual plane P or at a same height relative to each other, as schematically shown in FIG. 2B. In some other examples, first band guiding recess 208 and second band guiding recess 226 may be non-aligned, e.g., not lie within the same plane P. In some examples, first band guiding recess 208 and second band guiding recess 226 may have different or identical angular orientations relative to each other.

In some examples, one or both band guiding recesses 208 and 226 may have, respectively hook-shaped protrusion 230 and 232 extending, relative to the center C of band, from a distal surface of the recess towards the proximal surface of the same recess. This way, a track or suture holding element is formed in either recess facilitating guiding and form-fittingly retaining the band in place to prevent movement of the suture band in transversal directions X1 and X2.

In some embodiments, one or both band guiding recesses 208 and 226 may be elevated relative to band cutting recess 276, with respect to reference center C. For example, the inner surfaces of band guiding recesses 208 and 226 that are proximal to center C may be further away from the center C than the lowest point of U-shaped band cutting recess 276. In some examples, band cutting recess 276, which may be U-shaped, may extend from its lowest point to terminate, at either side of the U-shaped recess, at an edge that is formed by the lower surface of the corresponding band guiding recesses 208 and 226.

The shapes of any of the recesses described herein may vary from model to model and may be for example, slit shaped, cup shaped, “u” shaped.

The band guiding recesses of any of the devices described herein may be configured to hold two ends 212, 214 of a suture band.

Turning now to FIG. 2C, device 250 comprises two tracks or slots 258 and 259 which are of a width, height and spacing sufficient to permit the looping of the suture band through the slots.

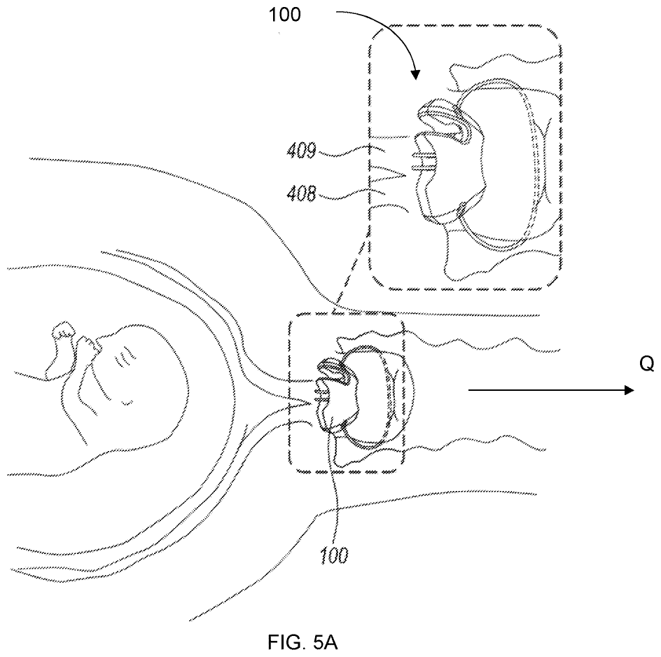

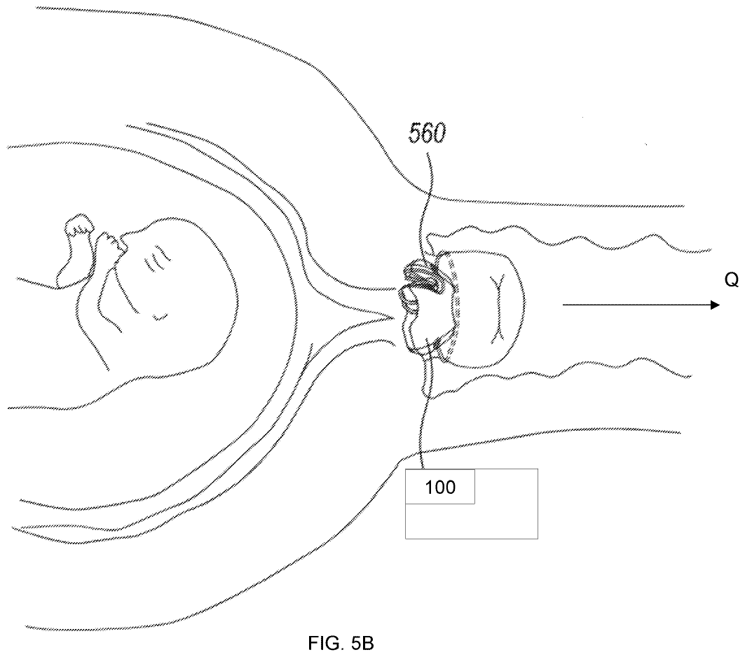

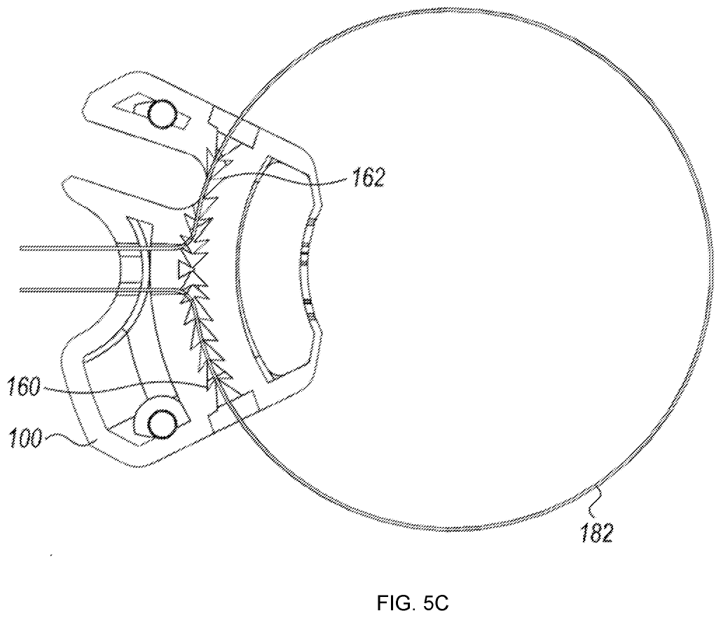

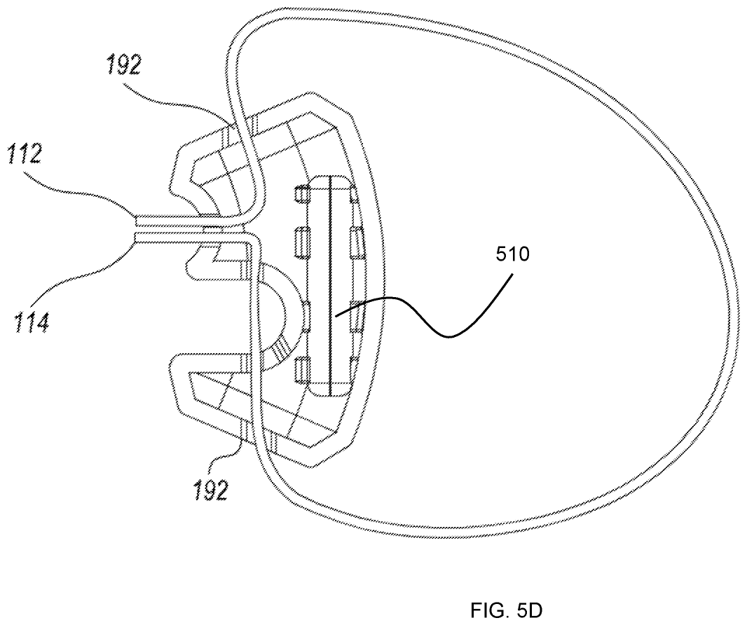

As mentioned herein, the device 200 may have two suture band holding elements 252 and 254, facing outward from an exterior side 270 of the device. The suture band may be glided in a first direction in between an inner suture band horizontal surface 260 and a second suture band upper holding element 254, over the band cutting recess 276 and into the first suture band holding element 252 into a track 258 terminating in slot 259. The first and second recesses may have suture band retainer elements 262 and 264, facing outwards and inwards, respectively. Alternatively, the suture band may be introduced into the device in the second direction opposite to the first direction. The suture band may then be cut to produce two ends 212, 214. Alternatively, it may be threaded from both sides to leave ends 212, 214 exposed in the first recess. A medical professional can then use the ends to tie a knot (560, FIG. 5B), when the device is set in a correct place and surrounds the cervix at the correct height thereof.

In some examples, an inner surface 280 of the inner part is may be arched or concave, serving as a completing a small part of an entire generally circular inner face of the suture band, constructed and configured to be held in place.

Reference is now made to FIG. 3, which is a simplified flowchart 300 of method for applying a cervix cerclage device to prevent loss of a pregnancy or a premature delivery in accordance with an embodiment.

Once a female human subject has been determined to be in need of a cerclage, she is brought into a clinic for a procedure. This may be before or in early pregnancy (such as weeks 10-18, commonly 14-16 of pregnancy).

A suture band 110, is introduced into a device 100, 200, as described hereinabove. The device and the suture band are introduced into a vagina in a device introducing step 302. This may be performed under local or general anesthetic. One advantage of the present device is that this procedure may be performed at a local clinic without needs for a surgeon.

In an affixing device step 304, the suture band is carefully introduced over a distal end of a cervix to ensure closure of the distal end of the cervix (see FIGS. 4A and 5A below).

In a device locking step 306, the suture band is tightened to prevent the cervix from opening and a knot is made using two ends of the suture band. Alternatively, there may be a knot in place and means to tighten/pull the band using the device. It should be noted that many alternative locking methods are deemed to be within the scope of the present invention, and that the examples discussed herein are by no means to be construed in a limiting manner. The female subject is then allowed to recover from the procedure and is typically checked using ultrasound to ensure that the fetus, placenta etc. are well placed and are in good shape (see FIGS. 4B and 5B below).

In a waiting step 308, the female subject waits, typically to 30-36 weeks of gestation. The subject is checked-up and asked to return to remove the device, again by a medical professional.

In a cutting suture band step 310, the professional cuts the suture band, such as at second recess 116 (FIGS. 1B), 226 (FIG. 2A) According to some embodiments, this may be performed by a gynecologist or other doctor, using outpatient services, rather than hospitalization. According to some embodiments, a nurse, midwife or other professional may perform this. Once the suture is cut, the device is released from surrounding the cervix and is easily removed from the female subject via her vagina, in a device removing step 312.

The fetus/baby is then ready for delivery within a matter of days/weeks and the female subject is free to leave the medical establishment.

FIG. 4A is a simplified transverse illustration of an open device inserted to engage a proximal end of a cervix, with respect to the subject′ amniotic sac 410, in accordance with an embodiment. Device 200 is inserted into a vagina 404 of a pregnant female. Typically, a fetus 420 grows within amniotic fluid 430 in the amniotic sac 410. Due to pressure forces/weak muscles 440 surrounded by tissue 450, a cervix 406 may be open at a distal end 408 thereof. This can lead to premature birth, miscarriage, early breaking of the waters (release of amniotic fluid) and other obstetric mishaps. Therefore, to prevent these mishaps, device 200 is introduced to surround the distal ends of the cervix. This can be seen in further detail in FIG. 4D.

FIG. 4B is a simplified transverse illustration of the closed (knotted) device inserted to engage a distal end of a cervix, in accordance with an embodiment. Device 200 sits at the distal ends of the cervix and a knot 460 of the suture thread/band 462 is seen exposed therefrom.

FIG. 4C is a simplified cross-sectional illustration of an open device, ready for use, in accordance with an embodiment.

FIG. 4D is simplified schematic illustrations showing a lower view, looking upwards of closed cerclage device 200 inserted to engage distal ends 408, 409 of a cervix, for preventing a miscarriage, in accordance with embodiments, showing a knot 480 of band 402.

FIG. 5A is a simplified transverse illustration of an open device 100 inserted to engage a distal end of a cervix, in accordance with an embodiment.

FIG. 5B is a simplified transverse illustration of a knot 560 exposed on a closed (knotted) device 200 inserted to engage a distal end of a cervix, in accordance with an embodiment.

FIG. 5C is a simplified cross-sectional illustration of an open device, ready for use, in accordance with an embodiment.

FIG. 5D is another simplified cross-sectional illustration of an open device, ready for use, in accordance with an embodiment.

The devices may further include one or more compartments 510 having openings for slow release of active pharmaceutical ingredients such as progesterone to delay labor.

Additionally or alternatively, in some embodiments, the device may be configured to thread the suture in longitudinal axial direction of the suture. In some other examples that thread may be introduced into holding/guiding structures from the side/transversely of a holding part of the device, and a cover may be then closed to assemble the device/fixate the sutures.

The references cited herein teach many principles that are applicable to the present invention. Therefore, the full contents of these publications are incorporated by reference herein where appropriate for teachings of additional or alternative details, features and/or technical background.

“Coupled with” means indirectly or directly “coupled with”.

Unless otherwise specified, the terms ‘about’ and/or ‘close’ with respect to a magnitude or a numerical value may imply to be within an inclusive range of −10% to +10% of the respective magnitude or value.

It should be appreciated that certain features, structures, characteristics, stages, methods, modules, elements, entities or systems disclosed herein, which are, for clarity, described in the context of separate examples, may also be provided in combination in a single example. Conversely, various features, structures, characteristics, stages, methods, modules, elements, entities or systems disclosed herein, which are, for brevity, described in the context of a single example, may also be provided separately or in any suitable sub-combination.

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable subcombination or as suitable in any other described embodiment, example or option of the invention. Certain features described in the context of various embodiments, examples and/or options are not to be considered essential features of those embodiments, unless the embodiment, example and/or option is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

As used herein, the phrase “A, B, C, or any combination of the aforesaid” should be interpreted as meaning all of the following: (i) A or B or C or any combination of A, B, and C, (ii) at least one of A, B, and C; and (iii) A, and/or B and/or C. This concept is illustrated for three elements (i.e., A, B, C), but extends to fewer and greater numbers of elements (e.g., A, B, C, D, etc.).

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

1. A device for locking during cerclage, the device comprising a passage to guide a suture therethrough and enable closure thereof around a distal end of a cervix in a female subject, the device adapted to remain engaged around the cervix over a period of time until the suture is cut.

2. The device according to claim 1, wherein the device further comprises a first recess in which two ends of the suture are knotted to form an exposed knot.

3. The device according to claim 1, further comprising a second recess in which a part of the suture is exposed to enable easy cutting thereof after the period of time.

4. The device according to claim 1, wherein the device can be introduced and removed from the cervix without surgery.

5. The device according to claim 1, further comprising holding means for said suture in place around the cervix.

6. The device according to claim 1, further comprising entry and exit orifices for said suture.

7. The device according to claim 1, further comprising an inner element, middle element and outer element.

8. The device according to claim 7, wherein said middle element holds the holding means.

9. The device according to claim 8, wherein the device is adapted to hold the suture in a generally circular shape around the cervix.

10. The device according to claim 1, wherein said suture is a suture band.

11. The device according to claim 1, wherein said device is polymeric and lightweight.

12. A method for performing cervical cerclage, the method comprising:
a. introducing a device according to claim 1 into a female subject;
b. enabling closure of a suture around a distal end of a cervix in the female subject; and
c. retaining said device around the cervix over a period of time until the suture is cut.

13. The method of claim 12, further comprising knotting two ends of the suture to form an exposed knot at a first recess.

14. The method of claim 12, further comprising exposing a part of the suture at a second recess to enable easy cutting thereof after the period of time.

15. The method of claim 12, further comprising introducing into and removing the device from the cervix without surgery.

16. The method of claim 12, further comprising holding said suture in place around the cervix by holding means.

17. The method of claim 12, further comprising entry and exit orifices for said suture.

18. The method of claim 12, further comprising holding the suture in a generally circular shape around the cervix.

19. The method of claim 12, wherein said suture is a suture band.

20. The method of claim 12, wherein said device is polymeric and lightweight.
